(19) United States
(12) Patent Application Publication
(10) Pub. No.: US 2026/0096575 A1
(43) Pub. Date: Apr. 9, 2026

(54) ENZYMATIC METHOD OF PRODUCING PLANT PROTEIN EXTRACT

(71) Applicant: NOVOZYMES A/S, Bagsvaerd (DK)

(72) Inventors: Zhen LONG, Beijing (CN); Mingzhu WANG, Beijing (CN); Yonghao CUI, Beijing (CN)

(73) Assignee: NOVOZYMES A/S, Bagsvaerd (DK)

(21) Appl. No.: 19/113,034

(22) PCT Filed: Sep. 28, 2023
(86) PCT No.: PCT/CN2023/122731

§ 371 Date: Mar. 19, 2025

(30) Foreign Application Priority Data
Sep. 30, 2022 (WO) PCT/CN2022/123078

(51) Int. Cl. A23J 1/14; C12N 9/80

(52) U.S. Cl. CPC A23J 1/148; C12N 9/80; C12Y 305/01002

(57) ABSTRACT

A method of producing plant protein extract, which comprises treating plant protein raw materials under alkaline conditions, recovering the protein and spray drying the recovered protein, wherein protein deamidase is added before alkaline treatment or during alkaline treatment or after alkaline treatment. The obtained plant protein extract has improved solubility which can be used in the food or beverage.

### REFERENCE TO A SEQUENCE LISTING

This application contains a Sequence Listing in computer readable form, which is incorporated herein by reference.

### FIELD OF THE INVENTION

The present invention relates to an enzymatic method of producing plant protein extract. Particularly, the present invention relates to a method for producing plant protein concentrate or plant protein isolate with a high solubility.

### BACKGROUND

The growing world population demands sustainably produced protein-rich foods. For a long time, legumes have been recognized as a valuable and low-cost source of high-quality protein products such as concentrates and isolates. Industrial scale production typically has applied soybean as the proteins source, whereas the most promising alternative to soy protein products are pea protein isolates. Plant proteins have a high potential value due to their characteristics (in particular, their suitability for health-oriented applications), and therefore their increased uses (range of applications) are also expected, for example, in food, beverages or the like. However, plant proteins often have a low solubility and poor functional properties. Deamidation is known to improve the solubility of plant proteins and as a consequence to improve the functional properties including foaming activity, foaming stability, emulsification activity and emulsification stability. This has been observed across several plant protein substrates including cereal proteins like oat, wheat, corn protein and legume proteins like e.g., soy and pea protein, coconut protein etc. Protein deamidases can be applied on almost all types of proteins (plant protein, animal protein, fermented proteins etc) where the enzyme will lower the isoelectric point of the protein, and will, when the proteins are applied at a pH above the isoelectric point, improve solubility, electrostatic repulsion, improve different types of functionalities like foaming, emulsification, water binding etc.

The market acceptance of use of plant-based protein in food or beverages is low, thus there remains a need for efficiently producing a plant protein extract having improved physical properties (in particular, solubility) to improve usefulness of the plant proteins.

### SUMMARY OF THE INVENTION

The inventors of the present invention have surprisingly found that during the process of preparing plant protein extracts from plant protein raw materials, some treatment processes and/or conditions directly and significantly influences the functional properties of the prepared plant protein extract, e.g., the solubility properties of the protein extract obtained, which makes the resulting protein extract much more suitable for different applications, such as foods and beverages, for the purpose of improving nutritive value.

Accordingly, in one aspect, the present invention relates to a method of producing plant protein extract, which comprises treating plant protein raw materials under alkaline conditions, recovering the protein and spray drying the recovered protein, wherein protein deamidase is added before alkaline treatment or during alkaline treatment or after alkaline treatment. Preferably, the plant protein extract is plant protein concentrate or plant protein isolate.

In other aspects, the present invention also relates to the plant protein extract obtained and the food or beverage containing the said plant protein extract.

### DETAILED DESCRIPTION OF THE INVENTION

The present invention relates to a method for producing plant protein extract, which comprises treating plant protein raw materials under alkaline conditions, recovering the protein and spray drying the recovered protein, wherein a protein deamidase is added before alkaline treatment or during alkaline treatment or after alkaline treatment. Preferably, the plant protein composition is plant protein concentrate or plant protein isolate.

Note that the singular forms “a,” “an,” and “the” include plural references unless the context clearly dictates otherwise.

Unless defined otherwise or clearly indicated by context, all technical and scientific terms used herein have the same meaning as commonly understood by one of ordinary skill in the art to which this invention belongs.

The term “about” indicates and encompasses an indicated value and a range above and below that value. In certain embodiments, the term “about” indicates the designated value plus or minus 10 percent, plus or minus 5 percent, or plus or minus 1 percent. In certain embodiments, the term “about” indicates the designated value plus or minus one standard deviation of that value.

In some embodiments of the present invention, plant protein raw materials are treated with a protein deamidase to significantly increase the solubility of the obtained plant protein extract.

Plant protein is normally applied in food ingredient as an extracted product from pre-processed state (i.e., gluten meal, seeds, or pulps). Plant protein product with higher protein content is typically preferred by the customer. Manufacturers thus tend to require high protein content plant protein extract in industrial methods. In some embodiments of the present invention, the plant protein composition is a plant protein concentrate or plant protein isolate. The term “plant protein concentrate” refers to a composition in which the protein content is increased as compared to that of pre-processed state (i.e., plant protein raw materials) due to extraction or concentration of the proteins. Typically such concentrate refers to a composition in which the protein content is of 40 to 80 percent (w/w), for example, some commercial soybean protein concentrate and pea protein concentrate contains 60-80 percent (w/w) protein content, while peanut protein concentrate, potato protein concentrate and rice protein concentrate are commercially available at around 50-70 percent (w/w) protein content. The term “plant protein isolate” refers to a composition in which the protein content is increased as compared to that of pre-processed state (i.e., plant protein raw materials) due to extraction or concentration of the proteins. Typically such isolate refers to a composition in which the protein content is of 80 percent (w/w) or more, for example, a range of 80-99 percent (w/w). Some commercial soybean protein isolate or pea protein isolate protein content is about 85% (w/w) or above, some purified soybean protein isolate or pea protein isolate can reach a protein content of 90% (w/w) or above.

In the present invention, the plant protein raw materials refer to plant materials containing proteins. The plant protein raw materials that are subjected to an enzyme treatment are not particularly limited. In general, the raw material used for plant protein extraction can be untreated plant raw material or raw material extracted by other components (i.e., oil, dextran, or starch). Some of these raw materials contain high oil content, which needs to be removed by pressing, organic extraction or carbon dioxide supercritical extraction to become raw materials for plant protein extraction, such as soybeans, peanuts, walnuts, perilla, sesame, cashews, hazelnuts, hemp seeds, etc. Some other plant raw materials are rich in starch, fiber and other substances, often through water washing, enzymatic degradation and other raw materials that can be used for plant protein extraction, such as corn, rice, wheat, oats, rye, quinoa, peas, mung beans, broad beans, potatoes and so on. Preferably, specific examples of the plant protein raw materials include peas, chickpea, soybeans, fava bean, lentil, oats, rye, barley, corn, amaranth, sesame, almond, peanut, cashew nut, hazelnut, pecan nut, macadamia nut, pistachio, walnut, Brazil nut, coconut, chestnut, pine nut, hemp seed, quinoa, rice and chia seed. Preferably, the plant protein raw materials are in the form of powder or flour.

In some embodiments of the present invention, the protein deamidase is a protein glutaminase. The protein glutaminase is known as a protein-glutamine glutaminase (also known as glutaminylpeptide glutaminase) activity, as described in EC 3.5.1.44, which catalyzes the hydrolysis of the gamma-amide of glutamine substituted at the carboxyl position or both the alpha-amino and carboxyl positions, e.g., L-glutaminylglycine and L-phenylalanyl-L-glutaminylglycine. Thus, deamidases can deamidate glutamine residues in proteins to glutamate residues and are also referred to as protein glutamine deamidase. Deamidases comprise a Cys-His-Asp catalytic triad (e.g., Cys-156, His-197, and Asp-217, as shown in Hashizume et al. “Crystal structures of protein glutaminase and its pro forms converted into enzyme-substrate complex”, Journal of Biological Chemistry, vol. 286, no. 44, pp. 38691-38702) and belong to the InterPro entry IPR041325.

Protein glutaminase activity was measured by using a fluorescence substrate comprising a glutamine residue and a fluorescence quenching group. The glutamine residue is converted to a glutamate residue by the deamidase activity, and the substrate is then cleaved by a glutamyl endopeptidase to remove the fluorescence quenching group.

In some embodiments of the present invention, the protein deamidase is a protein asparaginase. The protein asparaginase can directly act on an amide group of a side chain of an asparagine residue contained in a protein to release ammonia and thus converts the asparagine residue into an aspartate residue. In some embodiments of the present invention, as a protein deamidase, any one of the protein glutaminase and the protein asparaginase can be used, or both can be used in combination.

The types or origins of the protein deamidase used in the present invention are not particularly limited. Examples of the protein deamidase include protein deamidases derived from Chryseobacterium genus, Flavobacterium genus, Empedobacter genus, Sphingobacterium genus, Aureobacterium genus, or Myroides genus and the like, and commercially available protein glutaminases derived from Chryseobacterium genus. Protein glutaminases derived from Chryseobacterium proteolyticum are commercially available as, for example, Protein-glutaminase “Amano” 500 manufactured by Amano Enzyme Inc., and this commercially available products can be used. Preferred examples include protein deamidases derived from Chryseobacterium genus, preferred examples also include protein deamidases derived from Chryseobacterium proteolyticum. In some embodiments of the present invention, examples of the protein deamidase include protein deamidases derived from Chryseobacterium sp-62563, Chryseobacterium gambrini, Chryseobacterium culicis or Chryseobacterium defluvii or Chryseobacterium gleum. In some embodiments of the present invention, the protein deaminase can be derived from Chryseobacterium sp-62563 and disclosed as SEQ ID NO: 1 herein.

For purposes of the present invention, the sequence identity between two amino acid sequences is determined as the output of “longest identity” using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the nobrief option must be specified in the command line. The output of Needle labelled “longest identity” is calculated as follows:

(Identical Residues×100)/(Length of Alignment−Total Number of Gaps in Alignment)

In one embodiment the protein deamidase is selected from a polypeptide having at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 1.

In the context of the present invention, the term “variant” means a polypeptide having endopeptidase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more (e.g., several) amino acids, e.g., 1-5 amino acids, adjacent to and immediately following the amino acid occupying a position.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may affect the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for endopeptidase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Crushed materials or pulverized materials (powder) of the plant protein raw materials are generally subjected to the enzyme treatment so that the enzymatic reaction effectively proceeds. The protein deamidase can be added before step of alkaline treatment or during step of alkaline treatment or after alkaline treatment. Specifically, a protein deamidase is added to a suspension or solution of crushed materials or pulverized materials of plant protein raw materials to cause reactions.

In some embodiments of the present invention, the addition amount of a protein deamidase can be any amount of 0.001-100 mg enzyme protein per gram of plant protein raw material. Preferably, the ratio of 0.01-10 mg of enzyme protein per gram of plant protein raw material can be used, much preferably, the ratio of 0.1-1 mg of enzyme protein per gram of plant protein raw material can be used.

The plant protein raw material may preferably be incubated with the enzyme at a temperature of 5-80° C., preferably 10-65° C., such as 15-55° C. It is to be understood that the temperature may be adjusted during the incubation. The reaction time may be controlled, for example, within a range of 5 minutes to 1 days, preferably within a range of 10 minutes to 480 minutes, and more preferably within a range of 20 minutes to 120 minutes.

The conditions of treatment with a protein deamidase are not particularly limited as long as the treatment is efficient for the effect of the present invention, that is, increase and/or improvement in physical properties of the obtained plant protein extract. In preliminary experiments, plant protein raw material concentration, reaction temperature, reaction pH, reaction time, and the amount of the enzyme added (enzyme concentration) can be adjusted, and the optimal reaction conditions depending on the enzyme used can be established.

Plant protein extracts, such as plant protein concentrate or plant protein isolate can be extracted and/or concentrated by alkaline treatment and isoelectric precipitation method. When such a method is used, typically, the following steps are performed: (1) a step of adjusting the pH of an enzyme reaction solution and performing alkaline treatment to separate soluble components after the protein deamidase treatment, (2) a step of recovering proteins from the separated soluble components by an isoelectric precipitation method, and (3) a step of subjecting the recovered proteins to a neutralization process.

In some embodiments of the present invention, a method in which an alkaline treatment method and a membrane separation method are used in combination, or the like. In addition, as described above, a protein isolate is produced through steps, such as membrane concentration and the like, from a protein concentrate. Further, according to the production method of the present invention, a plant protein extract having a protein content within a range of 40 percent to 99 percent can be obtained.

In some embodiments of the present invention, during the step of alkaline treatment (preferably, alkali extraction), it is preferable that the treated or untreated plant protein raw materials can be dissolved in a ratio (w/w) of raw material powder:water in a scope range from 1:2 to 1:20 (corresponding to dry matter concentration at 5-50%), and any suitable alkali such as sodium hydroxide, potassium hydroxide and calcium hydroxide can be used for such pH adjustment. For example, 50% food grade sodium hydroxide solution (w/w) can be used to adjust the pH value of the slurry to achieve the required pH value for alkaline extraction, so as to dissolve the plant protein into the aqueous solution. In some embodiments, the extraction is performed at a pH value between about 7.0 and about 11.5, for example between about 7.5 and about 11.0, and preferably, the extraction is performed at a pH value between, about 7.5 and about 10, or about 8 and about 10, or between about 7.5 and about 9.0. In some embodiments, in some embodiments, the alkaline treatment is treated at 10° C. to 55° C. for a specified time (for example, 5 minutes to 24 hours, preferably 10 minutes to 2 hours).

Proteins are recovered from the separated soluble components by an isoelectric precipitation method. For example, when the soluble components are recovered as the supernatant after the centrifugal treatment, the pH of the supernatant (dilution or concentration may be performed before pH adjustment) is adjusted about 3 to 6 to precipitate proteins, and thereafter the precipitate is collected by centrifugal treatment. As used herein, the term “isoelectric point” refers to the pH at which a protein has the least net ionic charge. The pH of the isoelectric point of proteins and protein compositions can be determined by techniques known in the art. In a preferred embodiment, the pH of the solution containing protein is adjusted in the range of 3.0 to 6.0, preferably 4.5 to 5.5, preferably 4.5 to 5.0, and the pH can be adjusted by adding acid such as sulfuric acid or hydrochloric acid, citric acid or malic acid. At the isoelectric point, most proteins precipitate or aggregate.

After the isoelectric precipitation, the precipitated components can be separated by centrifugation, membrane separation, etc. In the process of centrifugation, desktop centrifuge, dish centrifuge, horizontal screw centrifuge or tube centrifuge can be used to recover protein by separating and collecting precipitate. In some embodiments, a desktop centrifuge is used for centrifugal treatment, the centrifugal speed is 4000 rpm, and the treatment time is 10 minutes. In some embodiments of the present invention, sterilization treatment is required for the recovered protein, typically, UHT, some technologies such as, pasteurization and/or flash sterilization can be used. In some preferred embodiments, the sample is pasteurized and heated in a water bath at 70-90° C. for about 10-30 minutes.

Typically, the precipitate collected is suspended in a suitable solvent (e.g., water is used), and thereafter alkali such as NaOH or sodium carbonate is added to neutralize the suspension. After the neutralization process, a treatment by concentration (e.g., membrane concentration, vacuum evaporation, or the like), drying methods (e.g., spray drying, freeze drying, or the like) is performed to obtain a plant protein extract.

In some embodiments of the present invention, during a step of spray drying, typically, dry hot air is used to evaporate the water in the uniformly atomized suspension containing plant protein extract, so as to achieve the purpose of separating the water from the dry matter in the suspension. Usually, the suspension containing plant protein extract and compressed air are sprayed and atomized together, and the water and dry matter are separated in the dry hot air environment. The dry matter is collected in the form of powder or particles under the double action of the induced draft fan and gravity. In some preferred embodiments, the atomized suspension with a dry matter concentration of 5-20% (w/w) is mixed with dry hot air at an inlet temperature of 130-170° C., and the temperature is gradually reduced to 80-100° C. in the process of water evaporation to reach the outlet, and then was collected. Usually, the water content of the collected plant protein extract is not higher than 15% (w/w), preferably, not higher than 10% (w/w).

Typically, during a step of freeze drying, the suspension containing plant protein extract needs to be pre-frozen, then the container containing the pre-frozen feed liquid is placed in a closed environment of a freeze dryer with a pressure of 0-200 Pa, and gradually thawed and dried. After sublimation, the water in the liquid is condensed in the cold trap of the freeze dryer and separated from the liquid to achieve the drying purpose. In some preferred embodiments, the concentration of the pre-frozen feed liquid is 5-20% (w/w), the pre-frozen temperature is −20° C., the cold trap temperature is −50° C., and the ambient pressure is 50 Pa. Usually, the feed liquid is freeze-dried under the above conditions and reaches a collectible state. Usually, the water content of the collected plant protein extracts is not higher than 10% (w/w).

### Use of Plant Protein Extract

The plant protein extract prepared by the invention exhibits a greatly improved solubility and other functional properties over known materials and can be incorporated in a wide variety of food materials to form superior protein fortified foods. For example, the plant protein extract can be directly used as food and has certain commercial value. In addition, it can also be used as food raw materials in food, beverages, fermented beverages, alcoholic beverages, condiments, baked foods, dairy products, health products, dietary supplements and other products. It can also be applied to animal feed and pet food. The plant protein extract of the invention may be used in various forms including but not limited to powder, granules, stick, baked biscuit or other products, pressed candy, soft capsule or hard capsule, soft candy or hard candy, gum or gel, paste, ice cream, jam, sauce, dried meat, alternative meat or vegetables, dairy or fermented dairy products.

The present invention is further described by the following examples that should not be construed as limiting the scope of the invention.

### EXAMPLES

### Strains

The Chryseobacterium sp-62563 strain was isolated from a soil sample collected in Sibhult, Sweden in September 2013.

### Enzyme

Protein glutaminase was derived from Chryseobacterium sp-62563 and disclosed as SEQ ID NO: 1.

### Example 1 Production of Plant Protein Isolate Using Protein Glutaminase Assay

### Pea Protein Content Determination:

The protein content in pea flour and pea protein isolate was determined by LECO FP-528 Nitrogen Analyzer. This method is to determine the nitrogen content based on combustion of the samples where, mainly CO₂, H₂O, NOx, N₂ are passed through different sorts of “filters” to exclude all but nitrogen. The nitrogen, in a helium carrier, is measured by a thermal conductivity cell. EDTA with nitrogen content of 9.57% was used as calibration standard. 6.25 was used as Nitrogen to protein index.

### Enzyme Protein (EP) Content Determination:

The enzyme protein (protein glutaminase) content was determined by Pierce BCA Protein Assay Kits, commercially available from Thermo Scientific.

### Production of Pea Protein Isolate

Pea flour from yellow pea was produced by milling of dehulled pea seeds for coarse milling followed by grinder for further milling to obtain a finer milled flour, the flour was sieved through a 0.5 μm sieve, protein content is 25.7% (w/w).

6 g of pea flour was suspended in water with a mass ratio of 1:6 (pea flour: water) in a shake flask. Then pH was adjusted to 6.5 and 9.0 by 6 mol/L NaOH for water soaking and alkaline soaking respectively, followed by the addition of protein glutaminase at a concentration of 0.4 (mg EP)/(g pea protein). Then, the flask containing pea flour was transferred to orbital shaker and incubated at 50° C./200 rpm for 1 hour. After that, the slurry was centrifuged at 4,000 rpm for 10 min. The supernatant was then collected. Subsequently, pH of the obtained supernatant was adjusted to 4.5 by 6 mol/L HCl. After another round of centrifugation at 4,000 rpm/10 min, the precipitate was collected and resuspended into water. After pH was adjusted to 6.8 with 6 mol/L NaOH, the slurry was dried in a freeze dryer or spray dryer to obtain pea protein isolate. The control was obtained in the same way as described above except that no protein glutaminase was added during soaking process.

### Drying Conditions

Freeze drying was performed in a Freeze dryer (LGJ-10C, Goring Technology Development (Beijing) Co., Ltd.). The freeze drying condition was: samples with a dry matter content of 10% (w/v) were dried in a freeze dryer with air pressure lower than 50 Pa until the water content of the sample was lower than 10% (w/w).

Spray drying was performed in a spray dryer (Mobile Minor™ 2000, GEA). The spray drying condition was set as follows: samples with a dry matter content of 8% (w/v) was fed by a constant-flow pump at speed 2.4 L/h. The inlet temperature of dry air was 170° C. with 0.2 MPa pressure, and the outlet temperature of plant protein powder and air was 73° C.

### Solubility Determination:

50 mg of different batches of pea protein isolate samples obtained were resuspended in 0.95 ml of 0.01 mol/L phosphate buffer. The phosphate buffer was prepared as following steps:
a) solution1: preparing 0.2 mol/L NaHPO₄·H2O solution, 35.01 g of NaHPO₄·H₂O was dissolved in distilled water, and add water until the solution volume is 1 L,
b) solution2: preparing 0.1 mol/L C₄H₂O₇·H2O solution. 21.01 g of C₄H₂O₇·H₂O was dissolved in distilled water, and add water until the solution volume is 1 L,
c) adjusting solution1 to final desired pH 7.0 by dropwise adding solution2 into solution1.

Such samples were vortexed and shaken at 50° C./1,200 rpm for 45 min. Then the mixture was centrifuged at 12,500 rpm for 15 min at room temperature. The soluble protein concentration in the supernatant was determined by Bicinchoninic Acid (BCA) Thermo Scientific™ kit. Bovine serum albumin was used as to build standard curve. Protein solubility was determined by dividing the soluble protein content of the supernatant by the total protein content in the sample (×100%).

### Relative Protein Solubility Determination:

The relative protein solubility of the samples was calculated by dividing the protein solubility of the control sample from the water soaking process by others (for both freeze drying and spray drying conditions). Therefore, the relative protein solubility of the control sample from the water soaking process was set to be 100%.

According to Table 1 and Table 2, adding protein glutaminase in both water soaking process and alkaline soaking process improved the relative protein solubility a bit at freeze drying condition comparing to the controls without protein glutaminase. However, when spray drying was employed, without protein glutaminase treatment, the batch relating to alkaline soaking process showed less relative protein solubility, whereas when we combined the alkaline soaking process with spray drying and protein glutaminase treatment, the relative protein solubility was highest, achieving 142%. These results suggested that during the process of preparing plant protein extract from plant protein raw materials, such treatments significantly influences the functional characteristics of the prepared plant protein extract, e.g., the solubility of the pea protein extract obtained, which makes the resulted pea protein extract much suitable for different applications, such as foods and beverages.

TABLE 1
Relative solubility of pea protein isolate obtained by freeze drying

| Batch | 1 | 2 | 3 | 4 |
|---|---|---|---|---|
| Soaking pH | 6.5 | 6.5 | 9.0 | 9.0 |
| mg EP/g pea protein | 0 | 0.4 | 0 | 0.4 |

| | |
|---|---|
| Drying method | Freeze dry |

| | | | | |
|---|---|---|---|---|
| Relative protein | 100% | 105% | 102% | 106% |
| solubility | | | | |

TABLE 2
Relative solubility of spray dried pea protein
isolate obtained by spray drying

| Batch | 5 | 6 | 7 | 8 |
|---|---|---|---|---|
| Soaking pH | 6.5 | 6.5 | 9.0 | 9.0 |
| mg EP/g pea protein | 0 | 0.4 | 0 | 0.4 |

| | |
|---|---|
| Drying method | Spray dry |

| | | | | |
|---|---|---|---|---|
| Relative protein | 100% | 108% | 87% | 142% |
| solubility | | | | |

1. A method of producing plant protein extract, which comprises treating plant protein raw materials under alkaline condition, recovering the protein and spray drying the recovered protein, wherein protein deamidase is added before alkaline treatment or during alkaline treatment or after alkaline treatment.

2. The method according to claim 1, wherein the protein deamidase is a protein glutaminase.

3. The method according to claim 1, wherein the plant protein raw material is one or two or more pulses, grains, or seeds selected from the group consisting of peas, chickpea, soybeans, fava bean, lentil, oats, rye, rice, barley, corn, amaranth, sesame, almond, peanut, cashew nut, hazelnut, pecan nut, macadamia nut, pistachio, walnut, Brazil nut, coconut, chestnut, pine nut, hemp seed, quinoa, and chia seed.

4. The method according to claim 1, wherein the protein deamidase is an enzyme derived from a Chryseobacterium genus microorganism, preferably derived from Chryseobacterium sp-62563.

5. The method according to claim 1, wherein the plant protein extract is plant protein isolate or plant protein concentrate.

6. The method according to claim 1, wherein the alkaline treatment is carried out under a condition with a pH of 7.5 to 11.

7. The method according to claim 1, wherein the step of recovering proteins is carried out by recovering proteins from separated soluble components by an isoelectric precipitation method.

8. The method according to claim 1, wherein the plant protein extract has an improved solubility.

9. A plant protein extract obtained by the method according to any one of claims 1 to 8.

10. A food or beverage containing plant protein extract obtained by the method according to any one of claims 1 to 8.


SEQUENCE LISTING (1 sequence)

SEQ ID NO: 1 - protein, 185 aa, Chryseobacterium sp-62563
AVSVIPNLATLNSLFAQIKNQACGTSTASSPCITFRYPVDGCYARAHKMRQILLNAGYDCEKQFVYGNLRASTGTCCVSWVYHVAILVSFKNASGVVEKRIIDPSLFSSGPVTDAAWRAACTNTSCGSASVSSFANTAGNVYYRSPSGSLLYDNNYVNTNCVLNIFSSLSGCSPSPAPSVASCGF